(19) United States
(12) Patent Application Publication
(10) Pub. No.: US 2023/0310707 A1
(43) Pub. Date: Oct. 5, 2023

(54) Porcine Scaffolds and Methods of Preparation

(71) Applicant: Convatec Traid Life Sciences, LLC., Memphis, TN (US)

(72) Inventors: Jon G. HARGIS, Memphis, TN (US); Kurt KLITZKE, Memphis, TN (US)

(21) Appl. No.: 17/288,041

(22) PCT Filed: Feb. 22, 2021
(86) PCT No.: PCT/US2021/019109

§ 371 Date: Apr. 23, 2021

Related U.S. Application Data
(60) Provisional application No. 62/979,731, filed on Feb. 21, 2020.

(51) Int. Cl. A61L 27/36; A61L 27/20; A61L 27/24

(52) U.S. Cl. CPC A61L 27/3633; A61L 27/20; A61L 27/24; A61L 27/3683

(57) ABSTRACT

Porcine scaffolds are provided that include decellularized, porcine placental extracellular matrix. Methods of preparing decellularized, porcine placental extracellular matrix as well as methods of treatment are also provided.

### CROSS-REFERENCE TO RELATED APPLICATIONS

The present application is a U.S. national stage application claiming priority to PCT/US2021/019109 filed Feb. 22, 2021 which claims priority to U.S. Serial No. 62/979,731 filed Feb. 21, 2020, the contents of which are each incorporated herein in their entirety.

### BACKGROUND

Extracellular matrices (ECMs) are the secreted molecules that create the microenvironment for cells and provide tissue with shape and strength (Brew, Dinakarpandian, & Nagase, 2000; Young, Holle, & Spatz, 2016). Biologic scaffolds created from extracellular matrix (ECM) are becoming increasingly common for the treatment of a variety of medical conditions (Hussey, Dziki, & Badylak, 2018). Commercially available scaffolds are created from a wide array of sources; multiple species and tissue types have been successfully processed into biologic scaffolds, including porcine small intestinal submucosa, bovine pericardium, porcine urinary bladder, and human dermis (Agmon & Christman, 2016).

In most commercially available ECMs, the structural component is primarily collagen, with the majority comprising type I collagen (Badylak, Freytes, & Gilbert, 2009). Additional fibril collagen species, types III, V and XI, and non-fibril collagen forms, types IV and VIII, will be present depending on the source material and tissue type (Theocharis, Skandalis, Gialeli, & Karamanos, 2016). In addition to the collagen content, ECMs can contain several adhesion molecules (Badylak et al., 2009) such as elastin, fibronectin, and laminin, which give each tissue type a unique ECM related to the tissue’s function. The third major structural component of ECM (Badylak et al., 2009) are proteoglycans (PG), a base protein bonded with one or more glycosaminoglycans (GAG). Some common extracellular proteoglycans are aggrecan, versican, and decorin but, like other structural molecules, proteoglycans content varies based on source material and tissue type (Theocharis et al., 2016).

When creating a biologic scaffold for healing damaged tissue, many factors influence the effectiveness of the final scaffold; source material, tissue type, quality of the tissue source, site of the tissue, age of the donor, recovery process, decellularization, manufacturing process and sterilization.

Placental membrane, with its critical role of protection and nourishment during fetal development, provides many unique properties that make it an ideal source tissue for a biologic scaffold (Shaifur Ra, Islam, Asaduzzama, & Shahedur R, 2015). To that end, human placental membrane (e.g., amniotic membrane and/or chorion tissue) has been used for various types of reconstructive surgical procedures since the early 1900′s. The membrane serves as a substrate material, more commonly referred to as a biological dressing or wound cover. Typically, human placental membranes are recovered after a cesarean section and are minimally processed, so that the manufacturing process does not alter the original relevant characteristics of the membrane relating to the membrane’s utility for reconstruction, repair, or replacement.

A primary function of the placental membrane is to provide a physiologic barrier to prevent desiccation (Mamede et al., 2012) and an immunological barrier for the fetus. Placental membrane tissue exhibits anti-microbial properties due to the presence of β-3 defensins that act to prevent microbial colonization of the epithelial surface (Chopra & Thomas, 2013; Niknejad et al., 2008). Anti-inflammatory properties, based on the presence of interleukin-4 (IL-4), interleukin-10 (IL-10), TIMP-1, TIMP-2 and TIMP-4 (Hortensius & Harley, 2016; Mamede et al., 2012), of placental membranes are also beneficial to tissue healing. Placental membranes have shown clinical evidence of epithelialization (Dua, Gomes, King, & Maharajan, 2004; Subrahmanyam, 1995; Ward & Bennett, 1984) and the potential to heal without scarring (Leavitt et al., 2016).

The quality of the source material for biologic scaffolds can prove challenging as all naturally-occurring materials have some inherent variability (Cardinal, 2015). This challenge is especially prevalent in human source materials. Variation in genetic expression among individuals has been well documented (Genomes Project et al., 2010; International HapMap et al., 2010), and that genetic variation has been demonstrated in tissue in single individuals (O′Huallachain, Karczewski, Weissman, Urban, & Snyder, 2012). In addition to the genetic variability, the tissue can also be influenced by a multiplicity of environmental and behavioral risk factors. For example, recovered human amniotic tissue can be affected by maternal lifestyle (Day et al., 2015). Increased expression of cytochrome P450 enzymes, a family of enzymes responsible for metabolizing toxic compounds, is a marker of oxidative stress in the human tissue (Strolin-Benedetti, Brogin, Bani, Oesch, & Hengstler, 1999). Human placental tissues have been shown to have increased cytochrome P450 levels in smokers (Huuskonen et al., 2016), drug users (Paakki et al., 2000), mothers with BMI >30 (DuBois et al., 2012), diabetics (McRobie, Glover, & Tracy, 1998) and alcohol users (Collier, Tingle, Paxton, Mitchell, & Keelan, 2002). Maternal and gestational age have been shown to alter expression of cytochrome P450 (Collier et al., 2002) and growth factors (Lopez-Valladares et al., 2010) in human placental tissues.

Despite the wide variability associated with human placental products, in the last decade, rising awareness of the healing properties associated with such products has led to an increasing demand, thus propelling market growth. There remains a need in the art, however, for wound-healing treatments with high clinical efficiency that overcome the inconsistencies of the commercially available human-sourced products.

### SUMMARY OF THE DISCLOSURE

The present disclosure is generally directed to porcine scaffolds and processes for producing such porcine scaffolds. The porcine scaffolds as disclosed herein exhibit various regenerative properties. The porcine scaffolds as provided herein can be cut into a variety of sizes as may be desirable for a particular application. The porcine scaffolds may also be placed on other areas of the body that have sustained damage but have not been subjected to surgical intervention.

A porcine scaffold is provided. According to one embodiment, the porcine scaffold includes decellularized, porcine placental extracellular matrix that includes at least about 0.5% w/w of hyaluronic acid based on the total weight of the porcine scaffold. According to one embodiment, the placental extracellular matrix is substantially devoid of intact cells. According to one embodiment, the placental extracellular matrix includes up to about 1200 ng dsDNA per mg of porcine scaffold. According to one embodiment, the porcine scaffold is formulated as a membrane-based construct. According to one embodiment, the placental extracellular matrix includes at least one placental membrane, at least one amnion membrane, at least one chorion membrane, or a combination thereof. According to one embodiment, the porcine scaffold includes at least about 1.0% w/w of hyaluronic acid based on the total weight of the porcine scaffold. According to one embodiment, the porcine scaffold includes at least about 1.5% w/w of hyaluronic acid based on the total weight of the porcine scaffold. According to one embodiment, the porcine scaffold includes at least about 2.0% w/w of hyaluronic acid based on the total weight of the porcine scaffold. According to one embodiment, the porcine scaffold includes at least about 2.5% w/w of hyaluronic acid based on the total weight of the porcine scaffold. According to one embodiment, the porcine scaffold retains at least about 50% w/w of any hyaluronic acid present in native porcine placental membrane. According to one embodiment, the porcine scaffold retains at least about 45% of any sulfated glycosaminoglycans present in native porcine placental membrane. According to one embodiment, the porcine scaffold retains at least about 66% of any elastin present in native porcine placental membrane. According to one embodiment, the porcine scaffold includes up to about 1650 ng of fibronectin per gram of porcine scaffold.

According to one aspect, a porcine scaffold is provided that includes decellularized, porcine placental extracellular matrix treated with a detergent and an alkali solution.

According to one aspect, a porcine scaffold is provided that includes decellularized, porcine placental extracellular matrix that includes up to about 85% w/w of total collagen based on the total weight of the porcine scaffold.

According to one aspect, a porcine scaffold is provided that includes decellularized, porcine placental extracellular matrix that includes up to about 10% w/w of elastin based on the total weight of the porcine scaffold.

According to one aspect, a porcine scaffold is provided that includes decellularized, porcine placental extracellular matrix that includes up to about 3% w/w of hyaluronic acid based on the total weight of the porcine scaffold.

According to one aspect, a porcine scaffold is provided that includes decellularized, porcine placental extracellular matrix that includes up to about 2% w/w of sulfated glycosaminoglycans based on the total weight of the porcine scaffold.

According to one aspect, a porcine scaffold is provided that includes decellularized, porcine placental extracellular matrix that includes up to about 99% w/w of total collagen, elastin, hyaluronic acid and sulfated glycosaminoglycans based on the total weight of the porcine scaffold.

According to one aspect, a porcine scaffold is provided that includes decellularized, porcine placental extracellular matrix that includes up to about 1650 ng fibronectin per gram of porcine scaffold.

According to one aspect, a porcine scaffold is provided that includes a surface defining one or more fenestrations.

A wound dressing is also provided. The wound dressing includes a porcine scaffold as provided herein. According to one embodiment, the wound dressing may include a surface defining one or more fenestrations.

According to one aspect, a method of preparing a porcine scaffold is provided that includes the step of processing the porcine placental membrane to form a porcine scaffold. According to one embodiment, the methods provided herein result in a porcine scaffold that retains at least about 50% w/w of any hyaluronic acid present in native porcine placental membrane. According to one embodiment, the methods provided herein result in a porcine scaffold that retains at least about 45% w/w of any sulfated glycosaminoglycans present in native porcine placental membrane. According to one embodiment, the methods provided herein result in a porcine scaffold that retains at least about 66% w/w of any elastin present in native porcine placental membrane. According to one embodiment, the methods provided herein result in a porcine scaffold that retains at least about 50% of sulfated and non-sulfated glycosaminoglycans present in native porcine placental membrane.

According to one embodiment, the step of decellularizing porcine placental extracellular matrix includes treating the porcine placental membrane with a detergent solution, the detergent solution including at least one protease enzyme. According to one embodiment, the detergent solution further includes at least one anionic detergent. According to one embodiment, the step of decellularizing porcine placental membrane includes treating the porcine placental membrane with a viral inactivation solution, the viral inactivation solution including at least one alkali solution. According to one embodiment, the alkali solution includes sodium hydroxide in an amount of about 1 mL to about 50 mL of about 0.1 M to about 3.0 M sodium hydroxide per gram of porcine placental membrane.

According to one aspect, a method of treating a defect is provided. The method of treating a defect includes the step of administering a porcine scaffold as provided herein to a defect. The defect may be located on or in a mammal in need of treatment. According to one embodiment, the defect is selected from a partial thickness wound, full thickness wound, pressure ulcer, venous ulcer, diabetic ulcer, chronic vascular ulcer, tunneled or undermined wound, surgical wound, wound dehiscence, abrasion, laceration, second degree burn, skin tear, and draining wound. According to one embodiment, the defect is a wound or ulcer.

According to another aspect, a method of treating a wound is provided that includes the steps of providing a powder-based construct as provided herein and placing the powder-based construct on or around a wound. According to one embodiment, the wound is an ulcer, abrasion or burn.

### BRIEF DESCRIPTION OF THE DRAWINGS

FIG. 1 shows a post-operative surgical wound dehiscence on a 63-year old male patient.

FIG. 2 shows the post-operative surgical wound of FIG. 1 after two weeks of standard care treatment.

FIGS. 3-7 show the post-operative surgical wound of FIG. 1 after five successive weekly treatments with one embodiment of a porcine scaffold.

FIG. 8 shows the post-operative surgical wound of FIG. 1 after the fifth application of the porcine scaffold.

FIG. 9 shows the post-operative surgical wound of FIG. 8 one month after the fifth application of the porcine scaffold.

FIG. 10 shows a post-operative surgical wound dehiscence on a 77-year old female patient.

FIG. 11 shows the post-operative surgical wound of FIG. 10 after four weeks of standard care treatment.

FIG. 12 shows the post-operative surgical wound of FIG. 10 after one week of treatment with one embodiment of a porcine scaffold.

FIG. 13 shows the post-operative surgical wound of FIG. 10 six weeks after the treatment with one embodiment of a porcine scaffold.

### DETAILED DESCRIPTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements.

As used in the specification, and in the appended claims, the singular forms “a”, “an”, “the”, include plural referents unless the context clearly dictates otherwise. As used in the specification, and in the appended claims, the words “optional” or “optionally” mean that the subsequently described event or circumstance can or cannot occur.

As used herein, the term “birth tissue” includes, but is not limited to, elements of mammalian birth tissue such as, for example, the placental membrane (amnion membrane and chorion membrane), Wharton’s jelly, umbilical cord, umbilical artery, umbilical vein, and amniotic fluid.

As used herein, the term “commercialized ECM product” is a commercialized extracellular matrix product composed of a porcine small intestinal submucosa source material.

As used herein, the terms “fenestration” and “fenestrated” may be used interchangeably and refer to placental membrane-based constructs which have been further modified to include at least one or a plurality of prearranged through-holes (fenestrations) in the construct. Such holes allow exudate to traverse through the construct . Further, the number and size of the holes is predetermined so as to ensure that the fenestrations are appropriately spaced to provide sufficient opportunity for the exudate produced by a wound to pass through the construct, while also maintaining sufficient construct surface area to effectively treat the wound.

As used herein, the term “placental membrane” refers to the full, intact placental membrane including the amnion and chorion layers that are obtained from a mammal such as, for example, a pig or human.

As used herein, the term “membrane” refers to at least one placental membrane, at least one amnion membrane, at least one chorion membrane or any combination thereof. The membranes as referred to herein may be obtained from a mammal such as, for example, a pig or human.

As used herein, the terms “pig” and “porcine” may be used interchangeably.

As used to herein, the terms “porcine scaffold” and “powder-based construct” refer to a construct that is applied onto or around an injured area of a mammalian body.

As used herein, the terms “defect” and “wound” may be used interchangeably and refer to an area in need of treatment such as an injured area of the mammalian body.

As used herein, the term “decellularization” refers to the process by which all or substantially all of the intact cells and nuclei are removed from the placental membrane, leaving a placental extracellular matrix derived from original placental membrane.

As used herein, the term “placental extracellular matrix” refers to the decellularized placental membrane whereby all or substantially all of the intact cells and nuclei are removed from the placental membrane, leaving a three-dimensional network consisting of extracellular macromolecules, such as collagen, elastin, glycosaminoglycans, laminin, and fibronectin, that provide both structural and biochemical support when used in the patient’s body.

As used herein, the term “scaffold” refers to a decellularized extracellular matrix structure that allows the patient’s cells to infiltrate and aid in the healing cascade and regeneration of damaged tissue. The scaffolds provided herein include extracellular matrices derived from birth tissue such as porcine placental membrane or other mammalian placental membrane.

As used herein, the term “native” refers to the condition of a tissue after procuring from a mammal but prior to being subject to the preparation steps provided herein.

The present disclosure provides an extracellular matrix scaffold that is prepared from mammalian birth tissue. The present disclosure particularly provides a porcine scaffold that is prepared from pig birth tissue. The methods and uses provided herein may be applied, however, to any mammalian-sourced birth tissue to form a scaffold suitable for regenerative purposes. Suitable mammals include, but are not limited to, human, bovine, equine, goat, or sheep.

Porcine placenta provides a unique source material for an extracellular matrix scaffold, while overcoming inherent challenges associated with human placenta. Social factors which seriously impact the availability and quality of human sourced tissue (e.g., obesity, tobacco, alcohol and drug consumption) are absent in porcine placenta. In contrast, purpose-bred sows have an entirely regimented life where the sows’ age, diet, exercise regimen/activity level, and health care are in the complete control of the breeder, thereby reducing the variability of the porcine placental starting material. For example, unlike the widely-adopted age criteria for human placental membranes which encompasses all women of childbearing years regardless of age, sows are bred from the age of one until about the age of six, which corresponds to a human age of range of 16 to 35. Sows birth the piglets at about 114 days of gestation. The combination of low maternal and gestational age lessen the chance of stress markers (e.g., cytochrome P450 enzymes) to be present in porcine tissue.

The porcine scaffolds and methods provided herein seek to address the unmet need in the current human placental membrane market by providing an alternate source material that overcomes inherent challenges associated with human placenta noted herein. Aside from the fact that the porcine-sourced material does not have the same age, health and lifestyle issues that may impact human products, the present porcine scaffolds allow host cells to infiltrate the scaffold and affected area (e.g., defect), deposit collagen, and easily and quickly remodel the defect.

The porcine scaffolds as provided herein may aid in the healing cascade or healing process of a mammalian defect such as a wound or ulcer. The porcine scaffolds may be fully resorbed by the mammal’s body during the healing process. Methods for aseptically processing placental membrane to prepare porcine scaffolds are provided. According to one embodiment, the porcine placental extracellular matrix placental are prepared from placental membranes that remain intact in that the placental membranes retain the amnion and chorion membrane layers and any intermediate layers. According to one embodiment, the placental membrane is processed in a manner such that all native layers are retained except for the Wharton’s Jelly.

The porcine scaffolds as provided herein may be formulated as a membrane-based construct. According to one embodiment, the porcine scaffold includes one or more layers of porcine placental membrane including the full, intact placental membrane or one or more layers of isolated amnion or chorion. According to one embodiment, the porcine placental membrane includes dehydrated, decellularized porcine placental extracellular matrix as provided herein. According to another embodiment, the porcine scaffold as provided herein may also be formulated as a powder, gel, liquid or spray.

According to one embodiment, when formulated as a membrane-based scaffold, the placental membrane chosen to be processed to form the scaffold may be treated to provide for the delivery of a variety of antibiotics, anti-inflammatory agents, growth factors and/or other specialized proteins or small molecules. In addition, the resulting membrane-based scaffold may be combined with or covered by a substrate (sterile gauze, sterile polymer material or other tissue or biomaterial) to increase the strength of the porcine scaffold for sutures or to increase the longevity of an implant.

A scaffold as described herein may be produced by processing mammalian birth tissue according to any or all of the steps provided herein as applied to birth tissue. According to a particular embodiment, a porcine scaffold as described herein may be produced by processing pig birth tissue according to the steps provided herein.

According to one aspect, a porcine scaffold is provided. According to one embodiment, the porcine scaffold includes decellularized, porcine placental extracellular matrix that includes one or more of collagen I, collagen III, collagen IV, elastin, laminin, fibronectin, hyaluronic acid and sulfated glycosaminoglycans. According to one embodiment, each of the one or more of collagen I, collagen III, collagen IV, elastin, laminin, fibronectin, hyaluronic acid and sulfated glycosaminoglycans is present in in an amount that is different from native porcine placental membrane that is not processed according to one or more of the processing steps provided herein.

According to one embodiment, the porcine scaffold as provided herein includes decellularized, porcine placental extracellular matrix that includes at least about 0.5% w/w of hyaluronic acid based on the total weight of the porcine scaffold. According to one embodiment, the porcine scaffold includes at least about 1.0% w/w of hyaluronic acid based on the total weight of the porcine scaffold. According to one embodiment, the porcine scaffold includes at least about 1.5% w/w of hyaluronic acid based on the total weight of the porcine scaffold. According to one embodiment, the porcine scaffold includes at least about 2.0% w/w of hyaluronic acid based on the total weight of the porcine scaffold. According to one embodiment, the porcine scaffold includes at least about 2.5% w/w of hyaluronic acid based on the total weight of the porcine scaffold. According to one embodiment, the porcine scaffold retains at least about 50% w/w of any hyaluronic acid present in native porcine membrane.

According to one embodiment, the placental extracellular matrix that forms the porcine scaffold is substantially devoid of intact cells. According to one embodiment, the placental extracellular matrix includes up to about 1200 ng dsDNA per mg of porcine scaffold.

According to one embodiment, the porcine scaffold is formulated as a membrane -based construct or scaffold. According to one embodiment, the placental extracellular matrix includes at least one dehydrated placental membrane, at least one dehydrated amnion membrane, at least one dehydrated chorion membrane, or a combination thereof. According to one embodiment, the placental extracellular matrix is chemically dehydrated.

According to one embodiment, the porcine scaffold retains at least about 45% of any sulfated glycosaminoglycans present in native porcine placental membrane. According to one embodiment, the porcine scaffold retains at least about 66% of any elastin present in native porcine placental membrane. According to one embodiment, the porcine scaffold includes up to about 1650 ng fibronectin per gram of porcine scaffold.

According to one embodiment, a porcine scaffold is provided that includes decellularized, porcine placental extracellular matrix treated with a detergent and an alkali solution. According to one embodiment, a porcine scaffold is provided that includes decellularized, porcine placental extracellular matrix that includes up to about 85% w/w of total collagen based on the total weight of the porcine scaffold. According to one embodiment, a porcine scaffold is provided that includes decellularized, porcine placental extracellular matrix that includes up to about 10% w/w of elastin based on the total weight of the porcine scaffold. According to one embodiment, a porcine scaffold is provided that includes decellularized, porcine placental extracellular matrix that includes up to about 3% w/w of hyaluronic acid based on the total weight of the porcine scaffold. According to one embodiment, a porcine scaffold is provided that includes decellularized, porcine placental extracellular matrix that includes up to about 2% w/w of sulfated glycosaminoglycans based on the total weight of the porcine scaffold. According to one embodiment, a porcine scaffold is provided that includes decellularized, porcine placental extracellular matrix that includes up to about 99% w/w of total collagen, elastin, hyaluronic acid and sulfated glycosaminoglycans based on the total weight of the porcine scaffold. According to one embodiment, a porcine scaffold is provided that includes decellularized, porcine placental extracellular matrix that includes up to about 1650 ng fibronectin per gram of porcine scaffold.

According to one embodiment, a porcine scaffold is provided that includes a surface defining one or more fenestrations.

A wound dressing is also provided. The wound dressing includes a porcine scaffold as provided herein. According to one embodiment, the wound dressing may include a surface defining one or more fenestrations. According to one embodiment, the wound dressing may be combined with or covered by a substrate or non-adherent secondary dressing (sterile gauze, sterile polymer material or other tissue or biomaterial) to increase the strength of the porcine scaffold for sutures or to increase the longevity of an implant.

A method of preparing a porcine scaffold. According to one embodiment, the method includes the step of processing the porcine placental membrane to form a porcine scaffold, wherein the porcine placental scaffold retains at least about 50% w/w of any hyaluronic acid present in native porcine placental membrane. According to one embodiment, the method includes the step of processing the porcine placental membrane to form a porcine scaffold, wherein the porcine scaffold retains at least about 45% w/w of any sulfated glycosaminoglycans present in native porcine placental membrane. According to one embodiment, the method includes the step of processing the porcine placental membrane to form a porcine scaffold, wherein the porcine placental scaffold retains at least about 66% w/w of any elastin present in native porcine placental membrane. According to one embodiment, the method includes the step of processing the porcine placental membrane to form a porcine scaffold, wherein the porcine scaffold retains at least about 40% w/w of any sulfated glycosaminoglycans present in native porcine placental membrane. According to one embodiment, the step of processing the porcine placental membrane to form a porcine scaffold includes treating the porcine placental membrane with a detergent solution, the detergent solution including at least one protease enzyme. According to one embodiment, the detergent solution further includes at least one anionic detergent. According to one embodiment, the step of processing the porcine placental membrane to form a porcine scaffold includes treating the porcine placental membrane with a viral inactivation solution, the viral inactivation solution including at least one alkali solution. According to one embodiment, the alkali solution includes sodium hydroxide in an amount of about 1 mL to about 50 mL of about 0.1 M to about 3.0 M sodium hydroxide per gram of porcine placental membrane. According to one embodiment, the porcine scaffold retains at least about 50% of sulfated and non-sulfated glycosaminoglycans present in native porcine placental membrane.

According to one embodiment, a method of preparing a porcine scaffold is provided. The method includes the step of collecting the birth tissue, including the umbilical cord, placental membrane (amnion and chorion membrane), and amniotic fluid from a female pig. According to one embodiment, the method includes the step of collecting the birth tissue, including the umbilical cord, placental membrane (amnion and chorion membrane), and amniotic fluid from a female pig. According to one embodiment, the female pig is not genetically modified to halt or reduce expression of the functional alpha-1,3 galactosyltransferase gene. According to one embodiment, the placental membrane includes the umbilical cord attached. Potential birth tissue donors are screened and tested to exclude any donors that may present a health risk. According to one embodiment, birth tissue is recovered from a full-term delivery of one or more offspring such as an infant or piglet(s). According to one embodiment, the method optionally includes the step of rinsing the birth tissue, including the umbilical cord and placental membrane (amnion and chorion membrane) by methods known to those skilled in the art. According to one embodiment, the method further includes the step of placing the birth tissue, including the umbilical cord and placental membrane (amnion and chorion membrane), in a transport container. According to one embodiment, the method further includes the step of placing the birth tissue, including the umbilical cord and placental membrane (amnion and chorion membrane), in a transport container containing transport solution.

According to one embodiment, the method optionally includes the step of freezing the umbilical cord and placental membrane by methods known to those skilled in the art. According to one embodiment, the umbilical cord and placental membrane may be kept frozen until further processing is needed. According to one embodiment, the method further includes the step of removing the frozen, bagged umbilical cord and placental membrane from the freezer and thawing in a refrigerator. According to one embodiment, the method further includes the step of thawing the umbilical cord and placental membrane at ambient temperature. According to one embodiment, the method optionally includes the step of placing any retained, frozen amniotic fluid in a container.

According to one embodiment, the method includes rinsing the umbilical cord and placental membrane with water. According to one embodiment, the method includes draining the umbilical cord and placental membrane. According to one embodiment, the method includes separating the placental membrane from the umbilical cord.

According to one embodiment, the method includes the step of dividing the placental membrane into pieces. According to one embodiment, a rotary cutter or other suitable cutter is used to cut the pieces. When formulated as a membrane-based construct, the birth tissue may be cut to various sizes, thickness, and shapes. The placental membrane pieces are preferably of sufficient size and shape to be applied onto or around a wound that is on or in a mammalian patient’s body. The placental membrane thickness may vary depending on application, the type of membrane and the number of membrane layers.

According to one embodiment, the method includes the step of removing Wharton’s jelly and excess fluids from the placental membrane to produce cleaned placental membrane.

According to one embodiment, the method includes the step of treating the placental membrane with a bioburden reduction solution. According to a preferred embodiment, the bioburden reduction solution is sodium chloride. According to one embodiment, the method includes the step of adding from about 1 mL to about 100 mL of 0.1 M to about 5 M sodium chloride solution per gram of placental membrane. According to one embodiment, the method includes the step of immersing the placental membrane in the sodium chloride solution from about fifteen minutes to about eight hours. According to one embodiment, the method includes the step of shaking the placental membrane in the sodium chloride solution from about fifteen minutes to about eight hours at about 20 RPM to about 100 RPM.

According to one embodiment, the method includes the step of decanting the sodium chloride. According to one embodiment, the method includes the step of rinsing the placental membrane with water. According to one embodiment, the placental membrane is rinsed one time with water. According to one embodiment, the rinsing step is carried out multiple times with water. According to one embodiment, the placental membrane is rinsed from about two times to about five times with water.

According to one embodiment, the method includes the step of placing the placental membrane in from about 1 mL to about 100 mL of a detergent solution. According to one embodiment, the detergent is present at a concentration of about 0.1% to about 10% w/v. According to one embodiment, the detergent solution includes at least one ionic detergent. According to a particular embodiment, the detergent solution includes at least one anionic detergent. According to a particular embodiment, the detergent solution includes at least one anionic detergent and at least one protease enzyme. According to one embodiment, the detergent solution contains phosphates, with a phosphorus content of about 7.5%. According to one embodiment, the detergent solution includes phosphates, carbonates, sodium linear alkylaryl sulfonate and at least one protease enzyme. According to one embodiment, the method includes the step of immersing the placental membrane in the detergent solution for from about fifteen minutes to about eight hours. According to one embodiment, the method includes the step of shaking the placental membrane in the detergent solution for from about fifteen minutes to about eight hours at about 20 RPM to about 100 RPM.

According to one embodiment, the method includes the step of decanting the detergent solution. According to one embodiment, the method includes the step of rinsing the placental membrane with water. According to one embodiment, the placental membrane is rinsed one time with water. According to one embodiment, the placental membrane is rinsed multiple times with water. According to one embodiment, the placental membrane is rinsed from about two times to about five times with water.

According to one embodiment, the method includes the step of treating the placental membrane with a viral inactivation solution, such as, for example, sodium hydroxide, hydrogen peroxide, ethanol or supercritical carbon dioxide. According to a preferred embodiment, the viral inactivation solution is sodium hydroxide. According to one embodiment, the method includes the step of adding or introducing from about 1 mL to about 50 mL of about 0.1 M to about 3.0 M sodium hydroxide per gram of placental membrane. According to one embodiment, the method includes the step of immersing the placental membrane in the sodium hydroxide for about 1 minute to about 120 minutes. According to one embodiment, the method includes the step of shaking the placental membrane in the sodium hydroxide for about 1 minute to about 120 minutes at about 20 RPM to about 100 RPM. The sodium hydroxide may then be decanted. According to one embodiment, the steps of adding sodium hydroxide, shaking and decanting may be repeated as many times as necessary to inactivate any viruses present in the placental membrane to produce a placental membrane that is substantially void of viruses. According to one embodiment, the steps of adding sodium hydroxide, shaking and decanting may be repeated once. According to one embodiment, the steps of adding sodium hydroxide, shaking and decanting may be repeated up to five times. According to a preferred embodiment, the method includes the step of adding or introducing from about 5 mL to about 15 mL of 0.25 M sodium hydroxide per gram of placental membrane. According to a preferred embodiment, the method includes the step of adding or introducing about 10 mL of 0.25 M sodium hydroxide for about 20 minutes, shaking, decanting and repeating the process one time. According to this preferred embodiment, the resulting porcine scaffold is substantially void of viruses, but much of the extracellular matrix composition is preserved, including a substantial percentage of the glycosaminoglycans. According to one embodiment, the method includes the step of rinsing the placental membrane with water.

According to one embodiment, the method includes the step of adding or introducing from about 1 mL to about 50 mL of buffer solution per gram of placental membrane. According to one embodiment, the method includes the step of immersing the placental membrane in the buffer solution. According to one embodiment, the method includes the step of shaking the placental membrane in the buffer solution for about 1 minute to about 120 minutes at about 20 RPM to about 100 RPM. The buffer solution may then be decanted. According to a preferred embodiment, the buffer solution is phosphate buffer solution. According to one embodiment, the method includes the step of measuring the pH of the placental membrane after buffer solution treatment. According to one embodiment, the steps of adding buffer solution, shaking and decanting may be repeated until the pH of the placental membrane is between about 6.8 and about 7.2.

According to one embodiment, the method includes the step of rinsing the placental membrane with water. According to one embodiment, the placental membrane is washed one time with water. According to one embodiment, the rinsing step is carried out multiple times with water. According to one embodiment, the placental membrane is rinsed from about two times to about five times with water.

When preparing a membrane-based construct, the placental membrane may be wet or dehydrated. According to one embodiment, the placental membrane may be dehydrated by any method known in the art, including, but not limited to, chemical dehydration (e.g., organic solvents), lyophilization, desiccation, oven dehydration and air drying. According to a preferred embodiment, the method includes the step of adding or introducing an alcohol to the placental membrane to cover the entire surface of the placental membrane (i.e., submerge the placental membrane). According to one embodiment, the method includes the step of adding or introducing from about 1 mL to about 100 mL of alcohol per gram of placental membrane. According to one embodiment, the placental membrane is fully submerged in the alcohol for from about ten minutes to about 24 hours. The alcohol may be any alcohol-safe and appropriate for contact with placental membrane. According to a particular embodiment, the alcohol is ethanol. According to one embodiment, the method includes the step of decanting or draining the alcohol from the placental membrane.

According to one embodiment, the method includes the step of spreading the placental membrane onto a drying table (e.g., a Delrin drying table). According to one embodiment, the placental membrane may be blotted with a micro fiber wipe or similar. The placental membrane may be spread in a manner so as to fully dehydrate the placental membrane while ensuring no wrinkles or bubbles are present.

When preparing a membrane-based construct, the method includes the step of cutting the placental membrane to a predetermined or desired size. According to one embodiment, the placental membrane is cut to size with a rotary cutter or other suitable instrument. According to one embodiment, the cuts are made with a scalpel blade.

According to another embodiment, the method includes the step of forming one or more (e.g., a plurality) of fenestrations in the placental membrane. Thus, the resulting scaffold includes a surface defining one or more fenestrations (e.g., through holes). According to one embodiment, the placental membrane may be fenestrated with a scalpel blade or other apparatus, with the one or more fenestrations appropriately spaced to provide sufficient opportunity for the exudate produced by a wound to pass through the placental membrane, while also maintaining sufficient placental membrane surface area to effectively treat a defect such as a wound or ulcer.

The processing methods provided herein result in a mammalian scaffold that includes a decellularized, placental extracellular matrix such as a decellularized, porcine placental extracellular matrix that is derived from placental membrane.

According to one embodiment, the method includes the step of placing the cut scaffold in one or more packaging materials.

According to one embodiment, the method includes the step of terminally sterilizing the packaged scaffold. According to one embodiment, the method of terminal sterilization may be e-beam irradiation, gamma irradiation, peracetic acid treatment, vaporized peracetic acid (VPA) treatment, any combination thereof, or any other terminal sterilization method known in the art.

According to another embodiment, the scaffold is formulated as a powder-based construct. When preparing the powder-based construct, the scaffold may be wet or dehydrated. According to one embodiment, the scaffold may be dehydrated by any method known in the art, including, but not limited to, chemical dehydration (e.g., organic solvents), lyophilization, desiccation, oven dehydration and air drying. In one embodiment, the method includes the step of cutting the scaffold into a plurality of strips. According to one embodiment, the strips of scaffold may then be placed into a mill and ground into a powder to form a powder-based construct. According to one embodiment, scaffold may consist of the whole placental membrane or a portion thereof, which may be placed into a mill and ground into a powder to form a powder-based construct. According to one embodiment, the powder-based construct may then be placed into appropriate containers or vials at a desired concentration. According to one embodiment, the method of preparing a powder-based construct includes the step of lyophilizing the milled/ground powder-based construct within the vials to remove residual moisture. Then, the vials containing the powder-based construct are terminally sterilized. According to one embodiment, the method of terminal sterilization may be e-beam irradiation, gamma irradiation, peracetic acid treatment, vaporized peracetic acid (VPA) treatment, any combination thereof, or any other terminal sterilization method known in the art.

A method of treating a defect is also provided. According to one embodiment, the method includes the step of providing a porcine scaffold as provided herein. The porcine scaffold is then administered (e.g., placed on or around) a defect. The defect may be a soft tissue defect including a wound such as, for example, a burn, cut, or abrasion. According to one embodiment, the defect is selected from a partial thickness wound, full thickness wound, pressure ulcer, venous ulcer, diabetic ulcer, chronic vascular ulcer, tunneled or undermined wound, surgical wound, wound dehiscence, abrasion, laceration, second degree burn, skin tear, and draining wound. The defect may also be any ulcer. According to one embodiment, the wound may be a surgical site anywhere on or in a mammalian body. The porcine scaffold may be placed over a surgical site or held in place by a patient’s musculature or skin. Sutures or staples may also be used to hold a membrane-based porcine scaffold in place. The porcine scaffold may be hydrated at the application site during treatment. The porcine scaffold may also be used as an implant. The porcine scaffold can also be used to cover an implant or other device that may be placed on or within a mammalian body.

According to one embodiment, the porcine scaffolds provided herein are useful in conjunction with general surgical procedures to aid in the healing cascade, reduce adhesions and reduce pain/inflammation. Such general surgical procedures include, but are not limited to, breast reconstruction, hernia repair/abdominal wall reconstruction/fascial reconstruction, and vascular bypass graft sites. According to one embodiment, the porcine scaffolds provided herein are useful as hemostasis or biological glues.

According to one embodiment, the porcine scaffolds provided herein are useful for the treatment, reduction and prevention of scar formation. Such scar formation may be the result of trauma or surgical procedure. The surgical procedure includes any procedure that may result in scarring. According to one embodiment, the porcine scaffolds provided herein are useful in neurological surgeries to aid in nerve regeneration or repair, act as a dural substitute, nerve conduit, nerve wrap or in conjunction with aneurysm repair. According to one embodiment, the porcine scaffolds provided herein are useful in orthopedic surgeries (e.g., sports-related injuries to muscle, ligament, and tendons; bone-related surgeries (e.g., spine), total joint replacement, laminectomies (anti-adhesion barrier), tendon/ligament repair, nerve repair, osteoarthritis, cartilage repair, and bone grafting). According to one embodiment, the porcine scaffolds provided herein are useful in colorectal surgery such as colon anastomoses or fistulae repair. According to one embodiment, the porcine scaffolds provided herein are useful in cosmetic surgeries as a dermal filler or to aid in skin wrinkle reduction, skin resurfacing, skin rejuvenation, and other cosmetic purposes.

According to one embodiment, the porcine scaffolds provided herein are useful in cardiovascular surgeries in conjunction with pericardial patch, heart valve leaflets, or vascular graft. According to one embodiment, the porcine scaffolds provided herein are useful in pulmonology for lung repair.

According to one embodiment, the porcine scaffolds provided herein are useful for the treatment and reduction of existing scars (e.g., scar revision). Particularly, the porcine scaffolds provided herein may be used to improve or reduce the appearance of scars, restores skin function and correct skin changes (disfigurement) such as those caused by an injury, wound, or previous surgery. According to one embodiment, the porcine scaffolds described herein can be used as a dressing to aid in the healing and prevention of scars such as those associated with cancer removal (e.g., Moh’s surgery).

According to one embodiment, the porcine scaffolds provided herein are useful for the treatment of defects in the ear, nose, mouth or throat such as in the treatment of oral fistulae or septum repair. In some embodiments, the porcine scaffolds provided herein are useful for the treatment of dental defects such as in the wrapping of dental implants, treatment of advanced gingival recession defect, soft palette reconstruction, periodontal defects, or guided tissue repair. According to one embodiment, the porcine scaffolds provided herein are useful for the treatment of ophthalmological conditions (e.g., ocular surface repair, keratitis, corneal ulcer/shield, or pteygium). According to one embodiment, the porcine scaffolds provided herein are useful for the treatment of various gynecological or urological applications such as in ureteral repair, hysterectomy, uterine fibrosis, urinary incontinence, or vaginal prolapse.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### Biochemical Analysis

An extensive biochemical analysis was conducted on one embodiment of a porcine scaffold as provided herein. The results indicated that the porcine scaffold was composed primarily of four major extracellular matrix components: collagen, elastin, hyaluronic acid and sulfated glycosaminoglycans (sGAGs). The specific amounts are listed in Table I below:

TABLE I

Porcine Scaffold Major ECM Components

| ECM Component | Amount (%w/w) |
|---|---|
| Total Collagen | 77% ± 8% |
| Elastin | 9% ± 1% |
| Hyaluronic Acid (HA) | 2% ± 0.5% |
| Sulfated GAGs (sGAG) | 1% ± 0.4% |

The Collagen I to Collagen III ratio and resulting Collagen I and III contents by mass in the porcine scaffold are provided in Table II below. For this analysis, the 77% total collagen amount provided in Table II was assumed to be comprised mainly of Collagen I and III.

TABLE II

Porcine Scaffold Collagen I:III Ratio

| | |
|---|---|
| Collagen I:III Ratio | 2.15 ± 0.11 |
| Collagen I | 53% (%w/w) |
| Collagen III | 24% (%w/w) |

ELISA testing was used to quantify Fibronectin found in the porcine scaffold (Example 5). The results indicated that the porcine scaffold contained an average amount of 1521.3 ± 112.8 nanograms of fibronectin per gram of porcine scaffold.

The biochemical composition analysis of one embodiment of the porcine scaffold manufactured by the methods provided herein indicated an extracellular matrix with optimal regenerative potential. One of ordinary skill in the art appreciates the evolution of regenerative wound care products from those manufactured from purified collagen to those manufactured from more complex extracellular matrix structures with biologically important extracellular matrix components intact (e.g., elastin, fibronectin, glycosaminoglycans, and other extracellular matrix molecules). Purified collagen products, by definition, (Cassel & Kanagy, 1949) have limited regenerative potential because increased collagen content invariably results in decreased non-collagenous extracellular matrix molecules with regenerative potential. Non-collagenous extracellular matrix molecules, such as elastin, fibronectin, hyaluronic acid, sulfated GAGs, and other molecules, can act in concert or alone to create a better wound healing environment. One of ordinary skill in the art appreciates that biomaterials derived from natural extracellular matrix sources which are very dense in collagen (e.g., tendon, dermis) are mechanically strong, but provide de minimis regenerative potential; and that biomaterials derived from natural extracellular matrix sources that are less dense in collagen with higher levels of non-collagenous components (e.g., urinary bladder, pericardium, peritoneum) tend to have more regenerative properties because of their higher levels of non-collagenous extracellular matrix components. To that end, the porcine scaffold as provided herein retains a high amount of non-collagenous extracellular matrix components, which aid in its wound healing capabilities, when compared to other commercialized biomaterials derived from natural extracellular matrix sources. For example, the average total collagen (the sum of soluble collagen and insoluble collagen) of the present porcine scaffold is approximately 77% w/w. The average total collagen of the commercialized ECM product is approximately 94% w/w. While both can be considered collagen biomaterials because they are each composed primarily of collagen, the fact that the present porcine scaffold contains less collagen, and more combined non-collagenous major extracellular matrix components, which include, hyaluronic acid, sulfated glycosaminoglycans, elastin, and fibronectin, indicates a more potent regenerative biomaterial compared to the commercialized ECM product.

### Non-Collagenous Molecules in Porcine Scaffold

Glycosaminoglycans (GAGs) are complex carbohydrates that are expressed ubiquitously and abundantly on the cell surface and in the extracellular matrix. The structural diversity of GAGs enables interaction with a wide variety of biological molecules and facilitates numerous functions in the extracellular matrix to regulate mechanical properties of a tissue including cell proliferation, cell adhesion, growth factor signaling, immune cell function, and collagen structure. Five linear, complex, polydisperse GAGs are made in mammalian systems. Hyaluronic acid (HA) is a non-sulfated GAG, while the sulfated GAGs include: chondroitin sulfate (CS), dermatan sulfate (DS), heparan sulfate (HS), and keratan sulfate (KS). The sulfated GAGs are bound to a protein core to form proteoglycans (PG), while the non-sulfated GAG, hyaluronic acid, can exist as a GAG chain.

### Hyaluronic Acid (Non-Sulfated GAG)

Hyaluronic acid is a natural and linear carbohydrate composed of beta-1,3-N-acetyl glucosamine and beta-1,4-glucuronic acid repeating disaccharide units with a molecular weight up to 6 MDa (WO 2010/003797 A1). Hyaluronic acid is distributed widely throughout connective, epithelial and neural tissues and is known for its ability to play an important role in promoting wound healing and tissue regeneration (Chen Wound Rep Reg 1999; Litwiniuk Wounds 2016; Kessiena Wound Rep Reg 2014). Hyaluronic acid is also involved in all stages of extracellular tissue repair: at the inflammatory, granulation, re-epithelialization and wound-healing stages (EP 1 948 200 B1). Hyaluronic acid is also known as hyaluronan, hyaluronate, or HA. The terms hyaluronic acid and HA may be used interchangeably herein.

Glycosaminoglycan (GAG) content of porcine placentas across the second half of gestation was found to be approximately 60 µg/mg (0.06 g/g), and at the latest stage of gestation, hyaluronic acid accounted for approximately 64% of the total GAG content (Steele 1980). Using this data, the average hyaluronic acid content of porcine placenta at term is typically about 38.4 µg/mg (0.038 g/g) and the remaining sulfated GAGs account for 21.6 µg/mg (0.022 g/g) at term.

The porcine scaffold as provided was shown to maintain at least about 0.02 gram of hyaluronic acid per gram of porcine scaffold (see Example 6). This measurement represents an about 50% retention in the hyaluronic acid content of the porcine scaffold post-processing. Thus, the porcine scaffold as provided herein retains at least about 50% w/w of any hyaluronic acid present in native porcine placental membrane. The retention of hyaluronic acid content in the porcine scaffold presents surprising results in view of the methods of preparation provided herein.

Decellularization of extracellular matrices can be achieved by various chemical treatments known in the art, some of which are known to remove GAGs during the decellularization process: alkalis/acids; non-ionic detergents (e.g., Triton X-100); ionic detergents (e.g., sodium dodecyl sulfate (SDS)); certain enzymes (e.g., nucleases) (Gilbert Biom 2006). According to the methods disclosed herein, the porcine scaffold may be prepared by a method including a step of treating porcine placental membrane with an ionic detergent. According to the methods disclosed herein, the porcine scaffold may be prepared by a method including a step of treating porcine placental membrane with an alkalis/acids (alkali-sodium hydroxide solution) treatment step. Despite these methods of preparation disclosed herein, a high content (at least about 50% w/w) of hyaluronic acid is preserved from the raw porcine placental tissue in the resulting porcine scaffold.

The porcine scaffold as provided herein includes significantly elevated levels of hyaluronic acid when compared to other commercially available extracellular matrix products. For example, the porcine scaffold as provided herein contains about ten (10) times more hyaluronic acid on a per gram (w/w) basis compared to other extracellular matrix products commercially available, including, for example, one sourced from porcine small intestinal submucosa (commercialized ECM product) and one sourced from human birth tissue. The commercialized ECM product contained approximately 0.002 g hyaluronic acid per gram of product (see Example 6); and the dehydrated human amnion/chorion membrane allograft also contained approximately 0.002 g hyaluronic acid per gram of product per the manufacturer’s published data (Lei Adv Wound Care 2016).

### Sulfated GAGs

Sulfated GAG side chains of proteoglycans are very important macromolecules in all phases of wound healing (Ghatak, S. et al.). During the proliferation phase of wound healing, fibroblasts and mesenchymal cells enter the inflammatory site of the wound in response to growth factors that are necessary for stimulation of cell proliferation. The fibroblasts synthesize collagen and proteoglycans, and such a process continues for several weeks with proportional increases of collagen. During this time, endothelial cells form capillaries, and the GAGs (hyaluronic acid, chondroitin sulfate (CS), and dermatan sulfate (DS)) levels also change. For the initial two weeks, hyaluronic acid is synthesized in large amounts by the fibroblasts, followed by increased levels of DS and CS proteoglycans. Gradually, when the proliferation of cells reaches a plateau, heparan sulfate (HS) proteoglycan levels are elevated in the wound. Sulfated proteoglycans with CS and DS assist in collagen polymerization, and HS proteoglycans on cells can create anchors to the surrounding matrix. Proteoglycan degradation by proteases in the wounds can release GAG-peptide fragments, which may modulate the wound healing process. For instance, CS and DS can regulate growth factor activity and may stimulate nitric oxide production, which, in turn, can modulate angiogenesis; whereas, HS can stimulate the release of IL-1, IL-6, PGE2, and TGF-β and contribute to the modulation of its proangiogenic effects in the tissues. Furthermore, sulfated GAGs as part of proteoglycans are thought to play a role in collagen fibril and fiber assembly (Michelacci, Y.M.) owing to the electrostatic interactions between positively charged sites on collagen molecules and the negatively charged GAGs.

The porcine scaffold as provided herein contains or otherwise maintains approximately 0.01 gram of sulfated GAGs per gram of porcine scaffold (see Example 7), which represents at least about 45% retention in the sulfated GAG content post-processing. Therefore, the porcine scaffold retains at least about 45% of any sulfated GAGs present in native porcine placental membrane. Similar to the hyaluronic acid retention outlined above, approximately half (e.g., at least about 50% w/w) of the sulfated GAG content is retained in the final porcine scaffold. Thus, processing porcine placental membrane according to the methods provided herein preserves at least about 50% w/w of the initial content of both sulfated and non-sulfated GAGs from the native porcine placental membrane in the final porcine scaffold.

### Elastin

Elastin is an extracellular matrix protein that lends elasticity and resilience to many tissue types and plays a critical role in wound healing, including inducing a range of cell activities, including cell migration and proliferation, matrix synthesis and protease production (Almine, Wise, & Weiss, 2012). Given its mechanical and signaling properties, elastin serves a multifunctional role in wound healing (Almine et al., 2013). The porcine scaffold as provided herein contains or otherwise maintains approximately 0.09 gram of elastin per gram of porcine scaffold, which represents at least about 66% retention in the elastin content post-processing (see Example 4). Therefore, the porcine scaffold retains at least about 66% of any elastin present in native porcine placental membrane.

### Fibronectin

Fibronectin is an adhesive glycoprotein that plays a crucial role in wound healing, particularly in extracellular matrix formation and in re-epithelialization (Lenselink, 2013). The porcine scaffold as provided herein contains or otherwise maintains approximately 1520 nanograms of fibronectin per gram of porcine scaffold (see Example 5). The commercialized ECM product contains approximately 139 nanograms of fibronectin per gram of commercialized ECM product, which equates to approximately 9% of the average fibronectin concentration found in the porcine scaffold. Thus, the porcine scaffold has over ten (10) times more fibronectin on a per ng/g (w/w) basis compared to the commercialized ECM product.

The porcine scaffold as provided herein retains a high amount of non-collagenous extracellular matrix components, which aid in its wound healing capabilities, when compared to other commercialized biomaterials derived from other natural extracellular matrix sources. The porcine scaffold as provided herein contains higher levels of several non-collagenous extracellular matrix molecules including, hyaluronic acid, elastin, and fibronectin, which suggests a more potent regenerative biomaterial compared to the commercialized ECM product.

### Decellularization of Porcine Scaffold

The proper decellularization of extracellular matrices is critical to the function of the final extracellular matrix product. The goal of decellularization is to remove the cells and eliminate residual genetic material, while preserving the extracellular matrix components and retaining the properties of the base tissue (Gilpin & Yang 2017). Incomplete decellularization has been shown to decrease the ratio of M2-activated macrophages to M1-macrophages when compared to more complete decellularization methods (Keane et al., 2012). Further studies have demonstrated that higher ratios of M2-activated to M1-activated macrophages are more promotive of immunomodulation, constructive mechanisms, and remodeling activities (Sicari et al, 2014).

One of ordinary skill in the art appreciates that cells can be removed from tissue using physical methods, chemical methods, enzymatic methods, protease inhibitors methods, or antibiotic methods. Regardless of the method of removal, a determination of the efficiency of decellularization is made. A common method for verifying the removal of cells is through the use of histologic staining. Hematoxylin and eosin (H&E) and DAPI (4′,6-diamidino-2-phenylindole) have been used successfully to quantify remaining cell and nuclear content, respectively, of processed tissues (Gilbert et al., 2006; Oliveira et al., 2013).

Cell debris testing was conducted on raw, washed porcine placentas in addition to the porcine scaffold in order to characterize the porcine scaffold manufacturing process’s ability to remove cellular material and generate a “clean” tissue.

Hematoxylin and eosin (H&E) staining was used to determine the presence of cells and cell debris. Also, 4′,6-diamidino-2-phenylindole (DAPI) staining was used for the assessment of intact nuclei. The starting raw porcine placental tissue contained abundant intact cells and nuclei throughout the tissue. In contrast, the porcine scaffold contained no visible intact cells and nuclei. Therefore, the porcine scaffold’s manufacturing process is an effective decellularization process.

Additionally, residual DNA/nucleic acid in the porcine scaffold was quantified using the IT PicoGreen Assay, yielding biochemical residuals amounting to miniscule amounts (1115.0 ± 137.0 ng/mg per porcine scaffold). Detailed test methods are discussed in Examples 2 and 3.

### Example 1

### Herovici Analysis - Semi-Quantitative Analysis of Collagen I to Collagen III Ratio

The objective of this study was to assess the ratio of Collagen I to Collagen III in: (i) porcine scaffolds prepared from three pig breeds (Breed 1, Breed 2, and Breed 3); and the commercialized ECM product. The Collagen I to Collagen III ratio was assessed by semi-quantitative analysis of the Herovici Stain. Testing was performed in accordance with the key requirements of Good Laboratory Practice regulatory standards (GLP).

### Methods

Samples from each test article were fixed in 10% neutral buffered formalin, embedded in paraffin, and 5 µm sections were cut onto slides using methods known in the art. Four 5 µm sections per test article were cut to each slide. The slides were stained. Two sets of the slides were stained using the Herovici protocol, each set stained as an independent batch. The ratio of pink (Collagen I) to blue (Collagen III) was quantified for four sections on each slide on the Nikon Eclipse E600 microscope and 20X objective using the Cri Nuance FX Multispectral Imaging System. The results are summarized in Tables III and IV.

### Results

TABLE III

| Sample | | Col I: Col III Ratio | SE |
|---|---|---|---|
| Porcine scaffold | Breed 1 | 2.27 | 0.49 |
| | Breed 2 | 2.04 | 0.35 |
| | Breed 3 | 2.12 | 0.26 |
| Commercialized ECM product | | 2.61 | 0.49 |

TABLE IV

| Sample | Col I: Col III Ratio | SE |
|---|---|---|
| Porcine scaffold: average of three breeds | 2.15 | 0.22 |
| Commercialized ECM product | 2.61 | 0.49 |

### Conclusion

The Herovici stain provided a method for a semi-quantitative analysis of the ratio of Collagen I to Collagen III in tissue samples (Turner et al, 2013). Herovici quantification of the test articles showed that the Collagen I: Collagen III ratio was 2.15 ± 0.22 for the porcine scaffolds; and 2.61 ± 0.49 for the commercialized ECM product.

A Kruskal-Wallis One Way Analysis of Variance on Ranks with All Pairwise Multiple Comparison Procedures (Dunn’s Method) was performed on the data and shows that for the Collagen I: Collagen III ratios there were no statistical differences between the commercialized ECM product and any of the three groups of porcine scaffolds prepared from Breed 1, Breed 2 and Breed 3.

### Example 2

### Assessment of Cell Debris

### Objective

The objective of this study was to assess the extent of cell debris present in: (a) porcine scaffolds manufactured from placenta from one of three breeds of pigs (Breed 1, Breed 2, and Breed 3) and (b) commercialized ECM product, with cellular material present in raw washed placentas from the three breeds (Breed 1, Breed 2, and Breed 3) serving as a tissue control. Testing was performed in accordance with the key requirements of Good Laboratory Practice regulatory standards (GLP).

### Methods

Samples from each test article were fixed in 10% neutral buffered formalin, embedded in paraffin, and 5 µm thick sections were cut onto slides. Two 5 µm sections per test article were cut to each slide. One slide from each sample was deparaffinized and stained for Hematoxylin and Eosin (H&E); one slide from each sample type was deparaffinized and stained with DAPI-Fluoromount-G Slide Mounting Medium (Southern Biotech # 010020), where DAPI (4′,6-diamidino-2-phenylindole) is a blue-fluorescent DNA stain that exhibits ~20-fold enhancement of fluorescence upon binding to AT regions of dsDNA. Slides were imaged on the Zeiss Observer Z1 microscope using the Axiocam MRc camera.

### Results

The test articles were stained with H&E and imaged at 32X magnifications to assess the presence of cells and cell debris. Additionally, the test articles were stained with DAPI and imaged at 10X and 32X to assess the presence of intact nuclei. Analysis of the H&E stained slides showed that: (i) the raw washed porcine placenta samples from the three pig breeds (Breed 1, Breed 2, and Breed 3) contained cellular material that appears as intact cells throughout the tissues, as would be expected for native placental tissue; and (ii) the porcine scaffold test articles manufactured from placenta from one of three breeds of pigs (Breed 1, Breed 2, and Breed 3) contained cellular material in certain areas but not throughout the tissue; and (iii) the commercialized ECM product contained cellular material throughout the tissue. While much of the cellular material observed in the commercialized ECM product appeared to be the size and shape expected for intact cells, the cellular material observed in the porcine scaffold test articles from Breed 1, Breed 2 and Breed 3 appeared to be smaller than intact cells.

Analysis of the DAPI stained slides shows that: (i) the raw washed placenta samples from the three breeds contained intact nuclei throughout the tissues as would be expected for native placental tissue, (ii) no intact nuclei were visible in the porcine scaffold test articles manufactured from the three breeds (Breed 1, Breed 2, and Breed 3); and (iii) the commercialized ECM product contained intact nuclei throughout the tissue.

The analysis of H&E and DAPI stained tissues indicated that the cellular material present in the porcine scaffold test articles was cellular debris and did not include intact cells, and that the commercialized ECM product contained intact cells throughout the tissue.

### Conclusion

When comparing cellular residuals that may pose an immunogenicity or inflammatory risk, the porcine scaffold is considered a safer material than the commercialized ECM product. The porcine scaffold presented no intact cells and nuclei, while the commercialized ECM product contained numerous intact cells and nuclei throughout its structure.

### Example 3

### Nucleic Acid Quantification and Analysis

### Objective

The objective of this study was to quantify the nucleic acids (by DNA content) in: (i) porcine scaffolds manufactured from placenta of one of three different breeds of pigs (Breed 1, Breed 2, and Breed 3).; and (ii) commercialized ECM product. Testing was performed in accordance with the key requirements of Good Laboratory Practice regulatory standards (GLP).

### Methods

DNA was extracted from samples following methods known in the art. Double-stranded DNA (dsDNA) was quantified using the Quant-IT PicoGreen Assay (Invitrogen, Carlsbad CA) according to manufacturer’s directions. All samples were assayed in triplicate. Furthermore, the test group samples were tested concurrently to ensure acceptability of the data due to the sensitivity of the assay. The results are summarized in Table V.

### Results:

### A. PicoGreen Quantification Results

TABLE V

| Sample | | ng DNA per mg dry weight | SD |
|---|---|---|---|
| Porcine scaffold | Breed 1 | 1020.05 | 128.01 |
| | Breed 2 | 1111.01 | 111.91 |
| | Breed 3 | 1213.94 | 111.06 |
| Commercialized ECM product | | 2322.59 | 41.75 |

### Conclusions

Each of the porcine scaffolds from one of three breeds of pigs (Breed 1, Breed 2, and Breed 3) contains approximately 1000 to 1200 ng dsDNA per mg dry weight. The commercialized ECM product contains over 2300 ng dsDNA per mg dry weight. The dsDNA content of each of the three porcine scaffold samples is approximately half of the dsDNA content of the commercialized ECM product.

These results, combined with the results from Example 2 (Assessment of Cell Debris), indicate that the porcine scaffold’s manufacturing process is a more effective decellularization process than the commercialized ECM product. The porcine scaffold is both cleaner and safer in relation to residual cellular and nucleic acid material in comparison to both the raw porcine placental material and the commercialized ECM product.

### Example 4

### Elastin Qualification

### Objective

The objective of this study was to measure the elastin content in: (i) porcine scaffolds manufactured from placenta of one of three different breeds of pigs (Breed 1, Breed 2, and Breed 3); (ii) raw washed placentas from the same three breeds; and (iii) commercialized ECM product. Testing was performed in accordance with the key requirements of Good Laboratory Practice regulatory standards (GLP).

### Methods

Each porcine scaffold test sample was prepared into a powder from a pool of porcine scaffolds derived from six individual sows from the same breed. Each raw washed placenta test sample was prepared into a powder from a pool of placentas derived from three individual sows from the same breed. Raw placenta material was prepared for testing from frozen placental material: the frozen porcine placentas were thawed, briefly rinsed in deionized water three times, trimmed, lyophilized, and finally powdered. All lyophilized material was powdered using a Wiley Mini Mill through a #40 mesh filter by methods commonly known in the art. One extract was prepared from each pooled sample and each extract was tested in triplicate.

All samples were quantified for elastin using the Fastin Elastin Assay Kit from Biocolor (Biocolor # F2000; Batch Code # BB087). Elastin was extracted from 6.3 mg of each sample. Sample extraction and quantification methods followed the manufacturer’s instructions. The results are summarized in Table VI.

### Results

TABLE VI

| Sample | | µg elastin per mg dry weight | SD |
|---|---|---|---|
| Porcine scaffold | Breed 1 | 65.67 | 9.42 |
| | Breed 2 | 97.07 | 5.07 |
| | Breed 3 | 94.88 | 4.22 |
| Native placenta | Breed 1 | 138.51 | 5.43 |
| | Breed 2 | 130.56 | 7.18 |
| | Breed 3 | 117.70 | 12.17 |
| Commercialized ECM product | | 78.50 | 2.73 |

### Conclusions

Elastin was quantified in (i) porcine scaffolds manufactured from three breeds (Breed 1, Breed 2, and Breed 3); (ii) raw washed placentas from the same three breeds; and (iii) commercialized ECM product. The porcine scaffolds contained between approximately 66 and 97 µg elastin per mg of tissue dry weight. The raw washed placentas contained between approximately 118 and 139 µg elastin per mg of tissue dry weight. It can be concluded that the porcine scaffolds retain a large percentage of the elastin present in the native tissue. The porcine scaffold as provided herein contains or otherwise maintains approximately 0.09 gram of elastin per gram of porcine scaffold, which represents at least about 66% retention in the elastin content post-processing.

### Example 5

### Fibronectin Quantification

### Objectives

The objective of this study was to quantify the concentration of Fibronectin in: (i) porcine scaffolds manufactured from one of three breeds of pigs (Breed 1, Breed 2, and Breed 3); (ii) raw washed placentas from the same three pig breeds; and (iii) commercialized ECM product. Testing was performed in accordance with the key requirements of Good Laboratory Practice regulatory standards (GLP).

### Methods

Each porcine scaffold test sample was prepared into a powder from a pool of porcine scaffolds derived from six individual sows from the same breed. Each raw washed placenta sample was prepared into a powder from a pool of placentas derived from three individual sows from the same breed. Raw placenta material was prepared for testing from frozen placental material: the frozen porcine placentas were thawed, briefly rinsed in deionized water three times, trimmed, lyophilized, and finally powdered. All lyophilized material was powdered using a Wiley Mini Mill through a #40 mesh filter. One extract was prepared from each pooled sample and each extract was tested in triplicate.

Samples were quantified for Fibronectin by enzyme-linked immunosorbent assay (ELISA). PBS extracts and urea-heparin extracts were prepared from all samples, and preliminary ELISAs were run to determine which preparation (PBS extraction or urea-heparin extraction) was more efficient for solubilization of each molecule to be quantified. Samples were then quantified from the appropriate extracts. Fibronectin was quantified from urea-heparin extracts of the samples using the LSBio Pig FN1 / Fibronectin ELISA, LSBio # LS-F8531. All quantification assays were run in triplicate and manufacturer’s instructions were followed. The results are summarized in Table VII.

### Results

### Quantification of Fibronectin (FN)

TABLE VII

| Sample | | Total ng FN per g dry weight | SD |
|---|---|---|---|
| Porcine scaffold | Breed 1 | 1408.78 | 70.88 |
| | Breed 2 | 1520.79 | 80.70 |
| | Breed 3 | 1634.43 | 85.13 |
| Native placenta | Breed 1 | 32667.85 | 1306.45 |
| | Breed 2 | 35452.94 | 1347.52 |
| | Breed 3 | 33187.87 | 873.04 |
| Commercialized ECM Product | | 139.16 | 9.78 |

### Conclusions

Fibronectin was quantified in: (i) porcine scaffolds from three breeds (Breed 1, Breed 2, and Breed 3); (ii) raw washed placentas from those three breeds; and (iii) commercialized ECM product.

The raw washed porcine placentas contained between 32,000 and 35,500 ng of FN per gram of tissue dry weight. FN remained present in the porcine scaffolds prepared from the three breeds (Breed 1, Breed 2 and Breed 3), ranging between 1,409 and 1,634 ng FN per gram of tissue dry weight. The commercialized ECM product contained 139 ng FN per gram of tissue of dry weight, approximately 9% of the average FN concentration in the porcine scaffolds. Thus, the porcine scaffold has over 10-fold more FN than the commercialized ECM product.

### Example 6

### Hyaluronic Acid Quantification

### Objective

The objective of this study was to measure the hyaluronic acid (HA) content in: (i) porcine scaffolds, each manufactured from placenta from one of three breeds of pigs (Breed 1, Breed 2, and Breed 3); (ii) commercialized ECM product; and (iii) porcine urinary bladder extracellular matrix (UECM) as an assay control. Testing was performed in accordance with the key requirements of Good Laboratory Practice regulatory standards (GLP).

### Methods

Each porcine scaffold test sample was prepared into a powder from a pool of porcine scaffolds derived from six individual sows from the same breed. One extract was prepared from each pooled sample and each extract was tested in triplicate.

All samples were quantified for HA using the Purple-Jelley Hyaluronan Assay Kit from Biocolor (Biocolor # H1000; Batch Code # BB031) and prepared according to the manufacturer’s instructions. The results are summarized in Table VIII.

### Results

TABLE VIII

| Sample | | mg HA per g dry weight | SD |
|---|---|---|---|
| Porcine scaffold | Breed 1 | 16.98 | 0.65 |
| | Breed 2 | 22.32 | 0.18 |
| | Breed 3 | 11.83 | 0.19 |
| Commercialized ECM product | | 1.58 | 0.05 |
| UECM (Control) | | 0.56 | 0.01 |

### Conclusions

The porcine scaffolds, the commercialized ECM product, and the UECM each contained measurable amounts of Hyaluronic Acid (HA).

The three porcine scaffolds, each prepared from a different breed of pig (Breed 1, Breed 2, and Breed 3) contained high levels of HA. The porcine scaffold prepared from Breed 1 contained 17.0 mg HA per gram tissue dry weight. The porcine scaffold prepared from Breed 2 contained 22.3 mg HA per gram tissue dry weight. The porcine scaffold prepared from Breed 3 contained 11.8 mg HA per gram tissue dry weight. These concentrations revealed that HA accounted for 1.7%, 2.2%, and 1.2% of the total dry weight of the porcine scaffolds from Breed 1, Breed 2, and Breed 3, respectively.

The commercialized ECM product contained 1.58 mg HA per gram tissue dry weight, indicating that HA accounted for 0.16% of the commercialized ECM product dry weight.

The UECM (control) contained 0.562 mg HA per g dry weight, indicating that HA accounted for 0.06% of the tissue dry weight.

The average amount of HA in the porcine scaffold was approximately 0.02 g of HA per gram of porcine scaffold, while the average amount of HA in the commercialized ECM product was approximately 0.002 g per gram of commercialized ECM product, indicating that the porcine scaffold had approximately ten (10) times more hyaluronic acid than the commercialized ECM product.

### Example 7

### Sulfated Glycosaminoglycan (GAG) Quantification

### Objective

The objective of this study was to measure the sulfated glycosaminoglycan (sGAG) content in: (i) porcine scaffolds, each manufactured from placenta from one of three breeds of pigs (Breed 1, Breed 2, and Breed 3); (ii) commercialized ECM product; and (iii) porcine urinary bladder extracellular matrix (UECM) as an assay control. Testing was performed in accordance with the key requirements of Good Laboratory Practice regulatory standards (GLP).

### Methods

All samples were digested at 50 mg/ml in 2 ml with 0.1 mg/ml proteinase K. Each porcine scaffold test sample was prepared into a powder from a pool of porcine scaffolds derived from six individual sows from the same breed. One extract was prepared from each pooled sample and each extract was tested in triplicate.

The sulfated glycosaminoglycans were quantified using the Blyscan Sulfated Glycosaminoglycan Assay Kit from Biocolor (Biocolor # B1000; Batch Code # BB053) following the manufacturer’s instructions. The results are summarized in Table IX.

### Results

TABLE IX

| Sample | | mg sGAGs per g dry weight | SD |
|---|---|---|---|
| Porcine scaffold | Breed 1 | 14.64 | 0.79 |
| | Breed 2 | 7.39 | 0.12 |
| | Breed 3 | 8.33 | 0.23 |
| Commercialized ECM product | | 11.93 | 0.28 |
| UECM | | 9.94 | 0.31 |

### Conclusions

The porcine scaffolds, each prepared from placenta from one of three breeds of pigs (Breed 1, Breed 2, and Breed 3), the commercialized ECM product, and the UECM each contained measurable amounts of sulfated glycosaminoglycans (sGAGs). The average concentration of sGAGs in the porcine scaffolds was 10.12 mg sGAGs per gram dry tissue weight. The concentration of sGAGs in the commercialized ECM product was 11.93 mg sGAGs per gram dry tissue weight. The concentration of sGAGs in the UECM was 9.94 mg sGAGs per gram dry tissue weight. Thus, the average amount of sGAGs in the porcine scaffold was approximately 0.01 g of sGAGs per gram of porcine scaffold, while the average amount of sGAGs in the commercialized ECM product was comparably 0.01 g of sGAGs per gram of commercialized ECM product.

### Example 8

### Collagen Quantification (Soluble - Insoluble)

### Objective

The objective of this study was to measure the collagen content in: (i) porcine scaffolds, each manufactured from placenta from one of three breeds of pigs (Breed 1, Breed 2, and Breed 3); (ii) commercialized ECM product; and (iii) porcine urinary bladder extracellular matrix (UECM) as an assay control. Both cold acid-pepsin soluble collagen and cold acid-pepsin insoluble collagen were quantified. Testing was performed in accordance with the key requirements of Good Laboratory Practice regulatory standards (GLP).

### Methods

Each porcine scaffold test sample was prepared into a powder from a pool of porcine scaffolds derived from six individual sows from the same breed. Cold acid-pepsin soluble collagen was extracted from each sample at 4° C. overnight at 10 mg/ml sample in 0.1 mg/ml pepsin / 0.5 M acetic acid and each extract was assayed in triplicate. One extract was prepared from each pooled sample and each extract was tested in triplicate. From the residues of each extract remaining after cold acid-pepsin extractions, collagen that was not cold acid-pepsin soluble was solubilized with heat and then was assayed in triplicate.

Collagen was quantified using the Sircol Dye binding method, with the collagen assay kits from Biocolor (Biocolor # S4000 = CLRS1000 (Sircol Soluble Assay) and CLRS2000 (Sircol Insoluble Assay) Batch Code # BB084). Extraction and quantification methods followed the manufacturer’s instructions. The results are summarized in Table X.

### Results

TABLE X

Collagen Expressed as a % of Tissue Dry Weight

| Sample | | Soluble Collagen | SD | Insoluble Collagen | SD |
|---|---|---|---|---|---|
| Porcine scaffold | Breed 1 | 1.60% | 0.05% | 68.27% | 1.04% |
| | Breed 2 | 2.22% | 0.02% | 72.04% | 0.77% |
| | Breed 3 | 2.73% | 0.06% | 83.06% | 0.26% |
| Commercialized ECM product | | 0.56% | 0.02% | 93.13% | 1.97% |
| UECM (control) | | 2.16% | 0.06% | 84.06% | 2.32% |

### Conclusions

In the porcine scaffold test samples, soluble collagen was approximately 1.6% to 2.7% of the total tissue dry weight, and insoluble collagen ranged from 70% to 85% of the total tissue dry weight. The commercialized ECM product had a lower concentration of soluble collagen and a higher concentration of insoluble collagen compared with the porcine scaffold samples.

### Example 9

### Immunolabeling for Collagen IV and Laminin

### Objective

The objective of this study was to evaluate the presence of Collagen IV and Laminin in: (i) porcine scaffolds, each manufactured from placenta from one of three breeds of pigs (Breed 1, Breed 2, and Breed 3); and (ii) raw washed placentas from those three breeds. Testing was performed in accordance with the key requirements of Good Laboratory Practice regulatory standards (GLP).

### Methods

Immunolabeling methods were used to identify the presence of Collagen IV and Laminin. Samples of test articles were hydrated in type 1 water for 1 hour and then fixed in 10% neutral buffered formalin (NBF) for at least 72 hours, embedded in paraffin, sectioned at 5 micron thickness, and mounted on glass slides. Antibodies used for the immunolabeling of Collagen IV and Laminin are summarized in Table XI.

TABLE XI

| | | |
|---|---|---|
| Molecule | Primary Antibody | Secondary Antibody |
| Collagen IV | Abcam ab6586 (rabbit polyclonal) | Sigma A0545 (HRP-conjugated anti rabbit IgG) |
| Laminin | Sigma L9393 (rabbit polyclonal) | Sigma A0545 (HRP-conjugated anti rabbit IgG) |

When each section of a test article was exposed to a block solution containing the primary antibody, a background control section on the same microscope slide was treated with the block solution only (“primary delete control”). All sections were then exposed to the secondary antibody diluted in the block solution. Following immunolabeling, slides were imaged on the Zeiss Observer Z1 microscope using the Axiocam MRc camera and the 20X objective.

### Conclusions

The presence of Collagen IV (Col IV) and Laminin (Lam) in: (i) porcine scaffolds manufactured from placenta from one of three breeds of pigs (Breed 1, Breed 2, and Breed 3); and (ii) raw washed placentas from those three breeds were assessed by immunolabeling. The immunolabeling study assessed the presence of Col IV and Lam that were recognized by the primary antibodies used for the immunolabeling and did not detect Col IV or Lam that had been changed during processing such that the epitopes recognized by the antibodies were no longer intact.

The immunolabeling results showed that Col IV was present in raw porcine placentas from all three breeds (Breed 1, Breed 2, and Breed 3) and was localized to the expected areas, specifically, areas rich in basement membrane. Additionally, Col IV was present in: the porcine scaffold prepared from Breed 1; the porcine scaffold prepared from Breed 2; and the porcine scaffold prepared from Breed 3.

The Lam immunolabeling results show that Lam was present in raw porcine placentas from all three breeds (Breed 1, Breed 2, and Breed 3) and was localized to the expected areas, areas rich in basement membrane and blood vessels. Additionally, Lam was present in: the porcine scaffold prepared from Breed 1; the porcine scaffold prepared from Breed 2; and the porcine scaffold prepared from Breed 3.

### Example 10

### Treatment of Post-Operative Incisional Wound Dehiscence

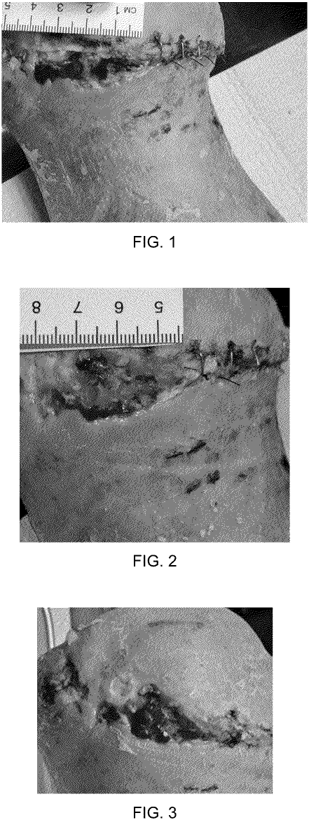

A 63-year old male patient presented with a post-operative surgical incisional dehiscence approximately three weeks after a Syme Amputation. Comorbidities included diabetes mellitus, coronary artery disease, and Stage 3 kidney disease. The patient presented with a post-operative surgical wound dehiscence with a length of 4 cm and a height of 1.5 cm (See FIG. 1). Initial treatment of the wound dehiscence involved standard of care treatment, including sharp debridement to clean margins and Betadine wet to dry bandage exchanges. Two weeks of successive treatments did not resolve the wound.

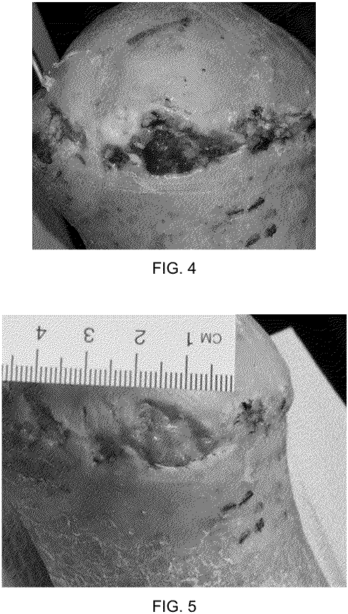

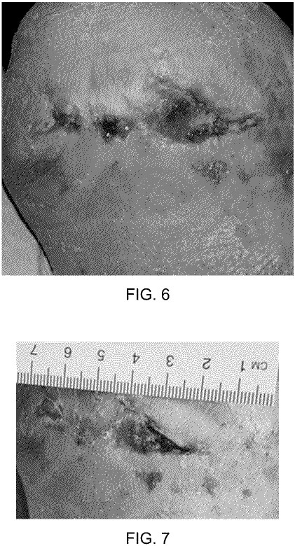

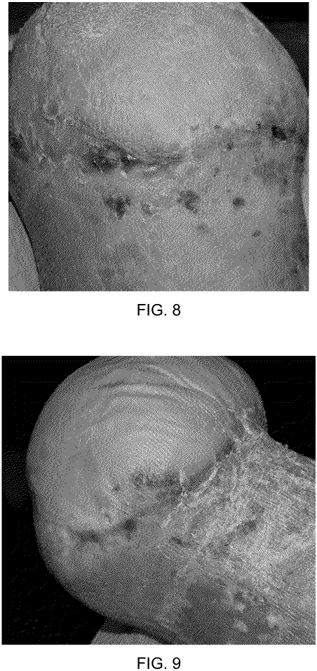

Following the two weeks of standard of care treatment, the wound underwent sharp debridement and cleaning leaving a wound 4 cm in length and 1 cm in height (See FIG. 2). The wound was covered with a porcine scaffold prepared according to the methods provided herein. A non-adherent secondary dressing and gauze was placed over the porcine scaffold. The patient underwent four more successive weeks of treatment, each involving debridement of the wound, application of the porcine scaffold, and placement of a non-adherent secondary dressing and gauze (See FIGS. 3-7). The wound exhibited a progression of healing during the five weeks of porcine scaffold applications. One week following the fifth application of the porcine scaffold, the wound exhibited re-epithelization and was deemed healed. (See FIG. 8). At the four-week follow-up appointment, the wound remained closed and healed (See FIG. 9).

### Example 11

### Treatment of Post-Operative Incisional Wound Dehiscence

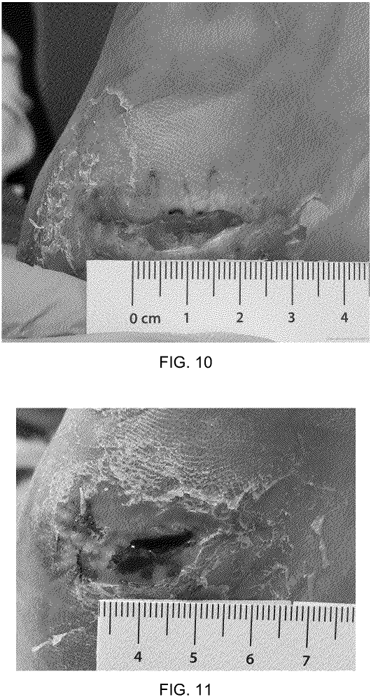

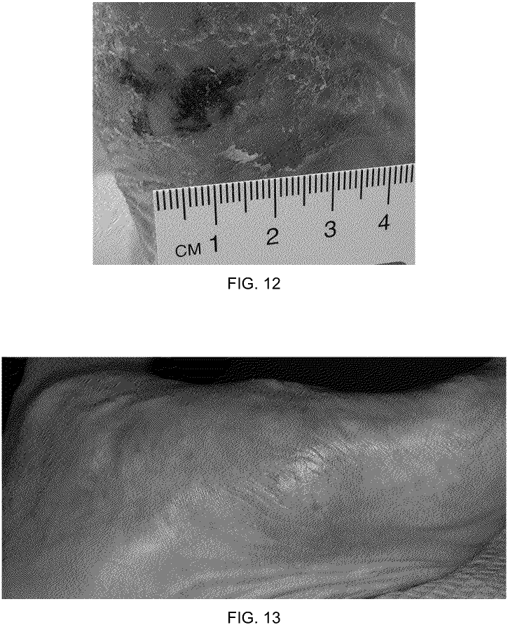

A 77-year old female patient presented with a post-operative surgical incisional dehiscence approximately two weeks after excision of a tarsal bone. Comorbidities included diabetes mellitus, diabetic neuropathy, and Charcot arthropathy. The patient presented with a post-operative surgical wound dehiscence with a length of 1 cm and a height of 0.2 cm (See FIG. 10). Initial treatment of the wound dehiscence involved standard of care treatment, including disinfection and sharp debridement to clean margins. Four weeks of successive treatments did not resolve the wound. Following the four weeks of standard of care treatments, the wound underwent sharp debridement and cleaning, leaving a wound 1 cm in length and 0.2 cm in height (See FIG. 11). The wound was covered with a porcine scaffold prepared according to the methods provided herein. A non-adherent secondary dressing and gauze was then applied over the porcine scaffold. The patient was placed in a walking boot and allowed to weight bear. At the one-week follow-up appointment, the wound was closed (See FIG. 12) and the patient transitioned a diabetic shoe with custom orthotics. At the six-week follow-up appointment, the wound remained closed and healed (See FIG. 13).

We claim:

1. A porcine scaffold comprising:
decellularized, porcine placental extracellular matrix,
wherein the porcine scaffold comprises at least about 0.5% w/w of hyaluronic acid based on the total weight of the porcine scaffold.

2. The porcine scaffold of claim 1, wherein the placental extracellular matrix is substantially devoid of intact cells.

3. The porcine scaffold of claim 1, wherein the porcine scaffold comprises up to about 1200 ng dsDNA per mg of porcine scaffold.

4. The porcine scaffold of claim 1, formulated as a membrane-based construct.

5. The porcine scaffold of claim 1, wherein the placental extracellular matrix comprises at least one placental membrane, at least one amnion membrane, at least one chorion membrane, or a combination thereof.

6. The porcine scaffold of claim 1, wherein the porcine scaffold comprises at least about 1.0% w/w of hyaluronic acid based on the total weight of the porcine scaffold.

7. The porcine scaffold of claim 1, wherein the porcine scaffold retains at least about 50% w/w of any hyaluronic acid present in native porcine placental membrane.

8. The porcine scaffold of claim 1, wherein the porcine scaffold retains at least about 45% of any sulfated glycosaminoglycans present in native porcine placental membrane.

9. The porcine scaffold of claim 1, wherein the porcine scaffold retains at least about 66% of any elastin present in native porcine placental membrane.

10. The porcine scaffold of claim 1, wherein the porcine scaffold comprises up to about 1650 ng fibronectin per gram of porcine scaffold.

11. A porcine scaffold comprising:
decellularized, porcine placental extracellular matrix according to claim 1, wherein the decellularized, porcine placental extracellular matrix is treated with a detergent and an alkali solution.

12. A method of preparing a porcine scaffold, the method comprising the step of: processing the porcine placental membrane to form a porcine scaffold,
wherein the porcine placental scaffold retains at least about 50% w/w of any hyaluronic acid present in native porcine placental membrane.

13. The method of claim 12, wherein the step of processing the porcine placental membrane to form a porcine scaffold includes treating the porcine placental membrane with a detergent solution, the detergent solution comprising at least one protease enzyme.

14. The method of claim 13, the detergent solution further comprising at least one anionic detergent.

15. The method of claim 12, wherein the step of processing the porcine placental membrane to form a porcine scaffold includes treating the porcine placental membrane with a viral inactivation solution, the viral inactivation solution comprising at least one alkali solution.

16. The method of claim 15, wherein the alkali solution comprises sodium hydroxide in an amount of about 1 mL to about 50 mL of about 0.1 M to about 3.0 M sodium hydroxide per gram of porcine placental membrane.

17. The method of claim 12, wherein the porcine scaffold retains at least about 50% of sulfated and non-sulfated glycosaminoglycans present in native porcine placental membrane.

18. A method of treating a defect, the method comprising the step of:
administering a porcine scaffold as provided in claim 1 to a defect on a patient in need of treatment.

19. The method of claim 18, wherein the defect is selected from a partial thickness wound, full thickness wound, pressure ulcer, venous ulcer, diabetic ulcer, chronic vascular ulcer, tunneled or undermined wound, surgical wound, wound dehiscence, abrasion, laceration, second degree burn, skin tear, and draining wound.

20. A wound dressing comprising a porcine scaffold of claim 1.

21. The wound dressing of claim 20, wherein the porcine scaffold includes a surface defining one or more fenestrations.
